(19) United States
(12) Patent Application Publication
(10) Pub. No.: US 2025/0114567 A1
(43) Pub. Date: Apr. 10, 2025

(54) Catheter Deflection Torque Control Mechanism

(71) Applicant: Cephea Valve Technologies, Inc., Abbott Park, IL (US)

(72) Inventors: Murrad Kazalbash, Santa Clara, CA (US); Austin Sherman, Sunnyvale, CA (US); Chase Carlson, San Jose, CA (US)

(73) Assignee: Cephea Valve Technologies, Inc., Abbott Park, IL (US)

(21) Appl. No.: 18/909,336

(22) Filed: Oct. 8, 2024

Related U.S. Application Data
(60) Provisional application No. 63/589,145, filed on Oct. 10, 2023.

(51) Int. Cl. A61M 25/01

(52) U.S. Cl. CPC A61M 25/0133

(57) ABSTRACT

A medical device delivery system includes a steering catheter having a flexible tip and a delivery mechanism comprising a coarse control and a fine control. A medical device delivery system includes a steering catheter having a flexible tip and a delivery mechanism comprising a variable torque mechanism.

### CROSS REFERENCE TO RELATED APPLICATIONS

The present application claims priority to U.S. Provisional Ser. No. 63/589,145, filed Oct. 10, 2023 the disclosure of which is hereby incorporated by reference in its entirety as if fully set forth herein.

### BACKGROUND OF THE DISCLOSURE

The present disclosure generally relates to devices, systems, and methods for delivering an intravascular medical device into a patient for implantation. More particularly, the present disclosure relates to devices, systems, and methods for steering a transcatheter delivery device of a collapsible prosthetic heart valve to a native heart valve annulus.

Intravascular medical devices that are collapsible can be delivered into a patient less invasively than devices that are not collapsible. For example, a collapsible prosthetic heart valve may be delivered into a patient via a tube-like delivery apparatus such as a catheter, a trocar, a laparoscopic instrument, or the like. Intravascular delivery can avoid the need for a more invasive procedure, such as an open-chest, open-heart surgery, and thereby reduce the risks, costs, and time associated with open-heart surgical procedures.

Intravascular delivery devices, such as a steerable catheter, may benefit from precise steering mechanisms to properly position the medical device within the patient. If precise steering maneuvers are not implemented, the delivery location may be unreachable, damage may result to the patient, or the medical device may not be able to be optimally positioned. These steering mechanisms may include cables that are fed through holes in a series of rings. An operator can adjust a distal end of the catheter by operating one or more controls attached to a handle or body of the steerable catheter. These controls tense and relax the cables to cause deflection in at least one direction of the distal end of the catheter.

A safe, accurate, and efficient delivery system and method for placing an intravascular medical device that addresses some or all of the foregoing concerns is described herein.

### BRIEF SUMMARY OF THE INVENTION

One aspect of the present disclosure provides a medical device delivery system comprising: a steering catheter having a flexible tip defining a deflection angle relative to the steering catheter; and a delivery mechanism configured to control the deflection angle of the flexible tip, the delivery mechanism comprising a coarse control over a first range of deflection angles and a fine control over a second range of deflection angles.

In one example, the coarse control comprises a lever configured to deliver a bolus of tension to the steering catheter.

In one example, the fine control comprises a knob configured to rotate a spool and collect a cable of the steering catheter.

In one example, the coarse control and the fine control operate independently.

In one example, the delivery mechanism comprises: a housing coupled to the lever by a first coupling point, the housing defining a pair of longitudinal slots configured to receive protrusions of the lever.

In one example, manual actuation of the lever causes the knob to slide along a path defined by the longitudinal slots.

In one example, the longitudinal slots are linear, curved, or curvilinear.

In one example, the second range of deflection angles of the flexible tip aligns with a patient anatomy.

In one example, the steering catheter is nested within a retractable outer sheath that is configured to deploy a prosthetic heart valve.

In one example, the prosthetic heart valve is a prosthetic mitral valve.

Another aspect of the disclosure provides a medical device delivery system comprising: a steering catheter having a flexible tip defining a deflection angle relative to the steering catheter; and a delivery mechanism configured to control the deflection angle of the flexible tip, the delivery mechanism comprising variable torque mechanism configured to deliver a first torque over a first range of deflection angles and second torque over a second range of deflection angles.

In one example, the variable torque mechanism comprises a spool having a plurality of distinct radiuses.

In one example, the spool is oval-shaped.

In one example, the steering catheter is operatively connected to a cable, wherein cable is collected upon the spool and tension is applied to the cable, causing deflection of the flexible tip.

In one example, the variable torque mechanism comprises: a conical cam defining a helical path; and a follower configured to act as a collection point for a cable relative to the helical path.

In one example, the helical path defines a plurality of radiuses between a first end and a second end of the conical cam such that the variable torque mechanism is configured to exert a variable torque on the steering catheter.

In one example, a cable is fixed permanently or semi-permanently to the follower such that the cable is collected or advanced relative to the conical cam, wherein the cable couples to the flexible tip.

In one example, as the spool is rotated, either or both of the spool and/or the follower move such that the follower is moved toward the second end of the conical cam.

In one example, the steering catheter is nested within a retractable outer sheath that is configured to deploy a prosthetic heart valve.

In one example, the prosthetic heart valve is a prosthetic mitral valve.

In one example, the variable torque mechanism comprises: an axle; a drive gear coupled to the axle; a derailleur assembly coupled to the axle; and a cassette including a plurality of gears.

In one example, the derailleur assembly includes an idler configured to move a chain from a first gear of the plurality of gears that applies the first torque to a second gear of the plurality of gears that applies the second torque

### BRIEF DESCRIPTION OF THE DRAWINGS

FIG. 1 is a perspective view of a catheter delivery device, according to one embodiment.

FIG. 2 is a cross-section view of the catheter of FIG. 1.

FIG. 3 is a cut-away view of a human heart.

FIG. 4 is a perspective view of the outer sheath of the catheter of FIG. 1.

FIG. 5 is an enhanced view of the outer sheath of FIG. 4 showing the outer sheath's inner components.

FIGS. 6A-C are schematic diagrams depicting a delivery system in various states of deflection and schematically represent torque relative to deflection angle.

FIGS. 7A-C are schematic diagrams depicting a desired delivery system in various states of deflection and schematically represent torque relative to deflection angle.

FIGS. 8A-D depict various stages of catheter deflection of a delivery mechanism according to one or more aspects of the disclosure.

FIG. 9A is a perspective view of a portion of delivery system according to another aspect of the disclosure.

FIG. 9B is a top perspective view of a portion of the delivery system of FIG. 9A.

FIG. 9Cs a side cross-sectional view of the portion of delivery system of FIG. 9A.

FIGS. 10A-C depict views of various stages of a steering operation of the delivery system of FIGS. 9A-C.

FIG. 11A is a perspective view of a portion of delivery system according to a further aspect of the disclosure.

FIGS. 11B-C are schematic representations of exerted torque of the delivery system of FIG. 11A.

FIG. 12 is a perspective view of a portion of delivery system according to another aspect of the disclosure.

FIGS. 13A-F are views of various stages of a steering operation of the delivery system of FIG. 12.

### DETAILED DESCRIPTION

As used herein, the term “inflow end,” when used in connection with a prosthetic heart valve, refers to the end of the heart valve through which blood enters when the heart valve is functioning as intended, whereas the term “outflow end,” when used in connection with a prosthetic heart valve, refers to the end of the heart valve through which blood exits when the heart valve is functioning as intended. For a prosthetic mitral valve, the inflow end is closest to the left atrium when the heart valve is implanted in a patient, and the outflow end is closest to the left ventricle when the heart valve is implanted in a patient. Further, when used herein in connection with a delivery device, the terms “proximal” and “distal” are to be taken as relative to a user operating the device in an intended manner. “Proximal” is to be understood as relatively close to the user and “distal” is to be understood as relatively farther away from the user. Also as used herein, the terms “substantially,” “generally,” and “about” are intended to mean that slight deviations from absolute are included within the scope of the term so modified.

In the description which follows, a delivery system and the components thereof are described in connection with the delivery, positioning, and deployment of a prosthetic mitral valve at the native mitral valve annulus. However, it is to be understood that the delivery system and components described also may be used to deliver, position, and deploy other prosthetic cardiac valves, such as the aortic valve, the pulmonary valve, and the tricuspid valve, as well as other medical devices. Exemplary prosthetic heart valves that can be used with the delivery system described herein include the expandable prosthetic heart valves described in U.S. Pat. Pub. No. 2016/0158000; in U.S. Pat. No. 8,870,948; and in PCT Pub. No. WO 2016/183526, the disclosures of all of which are hereby incorporated by reference herein.

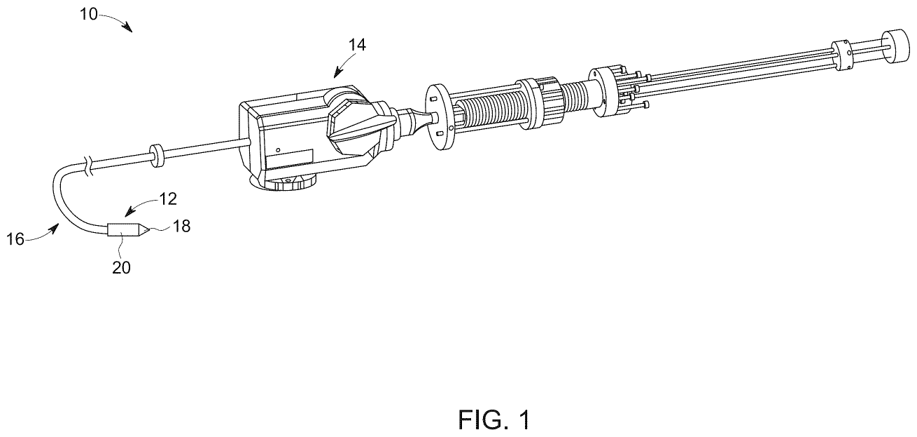

FIG. 1 illustrates a perspective view of a delivery system 10 for delivering, positioning, and deploying an exemplary prosthetic heart valve 20 in a patient, and then extracting a distal end 12 of the delivery system 10 out of the patient. It should be understood that, in FIG. 1, the prosthetic heart valve 20 is positioned within a valve cover of the delivery system 10, and thus the lead line for part number 20 points to the position of the collapsed prosthetic heart valve, which is not visible in FIG. 1 due to it being covered. Delivery system 10 generally includes a handle assembly 14 and a catheter assembly 16. Catheter assembly 16 extends from a proximal end of handle assembly 14. The distal end of catheter assembly 16 includes an atraumatic tip 18 and a plurality of catheter and/or hypotube components that are longitudinally slidable relative to each other and that provide different functionality during operation of delivery system 10 to enable effective delivery and deployment of a prosthetic heart valve 20, such as a prosthetic mitral valve.

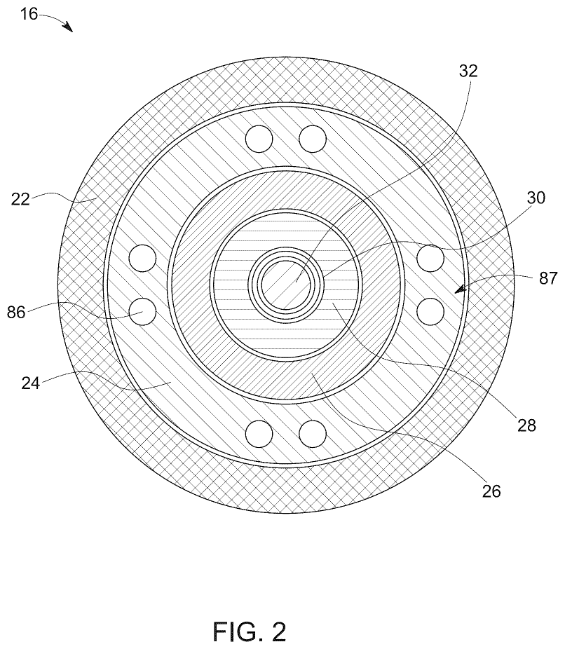

FIG. 2 shows a cross-sectional view of catheter assembly 16. Catheter assembly 16 includes the components of an outer sheath 22, a steering catheter 24, an extension catheter 26, a suture catheter 28, and a nosecone catheter 30, each catheter concentric and nested sequentially within another. As illustrated, nosecone catheter 30 has a lumen sized to receive a guidewire 32 therein. Each of these components is described in further detail below. It should be understood that the illustration of FIG. 2 is of a system with two-way steering, but the disclosure provided herein may apply to systems with one-way steering, or more than two-way steering.

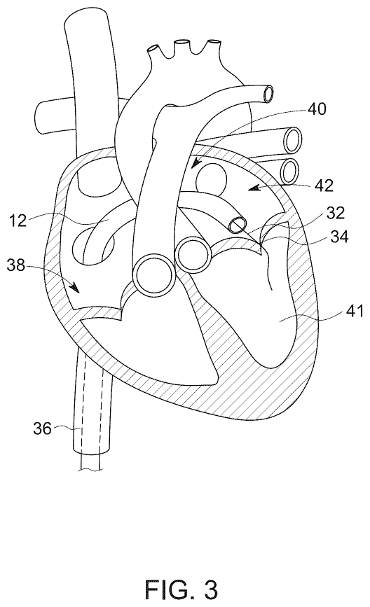

FIG. 3 shows a schematic representation of a patient's heart and a delivery route that may be followed by catheter assembly 16 to reach the native mitral valve annulus 34. Using a transfemoral approach, catheter assembly 16 may be inserted into the patient's femoral vein and advanced through the inferior vena cava 36 to the right atrium 38. Catheter assembly 16 is then advanced through a puncture made in atrial septum 40, into left atrium 42 and to the native mitral valve annulus 34.

In other implementations, such as for a procedure associated with a tricuspid (i.e. right atrioventricular) valve, catheter assembly 16 may be advanced through the inferior vena cava 36 and into the right atrium 38, where it may then be positioned and used to perform the procedure related to the tricuspid valve. Although many of the examples described herein relate to medical device delivery at the native mitral valve annulus 34, one or more embodiments may be utilized in other cardiac procedures, including those involving the tricuspid valve or other cardiac valves.

Although one preferred method for accessing a targeted cardiac valve annulus is a transfemoral approach, it will be understood that the embodiments described herein may also be utilized where alternative approaches are used. For example, embodiments described herein may be utilized in a transjugular approach, transapical approach, transradial approach, or other suitable approaches to the targeted anatomy. For procedures relating to the mitral valve or tricuspid valve, the delivery of the prosthetic heart valve or other medical device is preferably carried out from an atrial aspect (i.e., with the distal ends of catheter assembly 16 positioned within the atrium superior to the targeted valve). The illustrated embodiments are shown from such an atrial aspect. However, it will be understood that the delivery of the medical devices described herein may also be carried out from a ventricular aspect. In some circumstances, it is preferable to use a delivery route that avoids the requirement of making an incision in the chest and puncturing the left or right ventricle to access the desired valve being replaced. In other words, intravascular routes, such as via the femoral vein, may be less traumatic as they do not require chest incisions or puncturing the left or right ventricle of the heart.

Additional details regarding delivery systems and devices that may be utilized in conjunction with the components and features described herein are described in U.S. Patent Publication Nos. 2018/0028177, 2018/0092744, and 2020/0155804, the disclosures of which are hereby incorporated by reference herein.

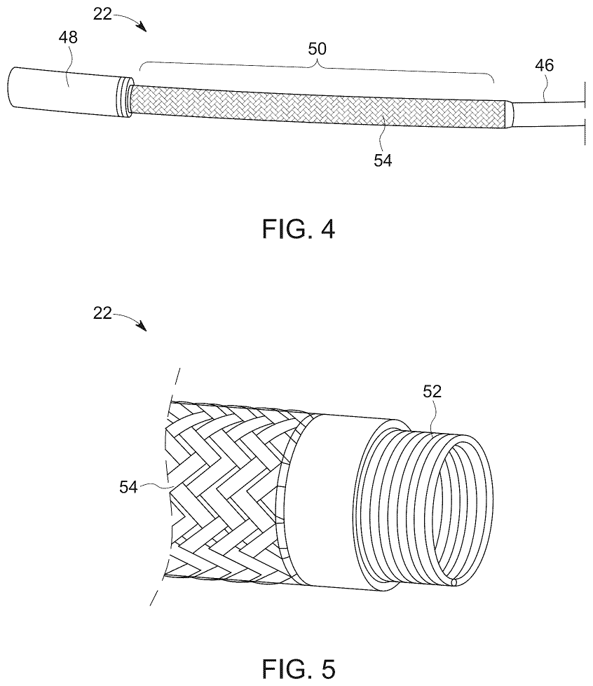

As shown in FIG. 4, a distal portion of outer sheath 22 is a bending portion 50 extending from the proximal portion to the distal end 48 of the outer sheath 22. The distal end 48 may be referred to as a valve cover as it may function to maintain a prosthetic heart valve in a collapsed condition during delivery. The distal portion may have a sufficient length to surround and extend along the portion of catheter assembly 16 that is designed to bend and deform to navigate through a patient's vasculature and heart to reach the mitral valve annulus for deployment of the prosthetic heart valve.

As shown in FIG. 5, bending portion 50 may have an inside layer formed from a coiled wire and an outer braided layer covering the coiled layer. Coiled layer 52 may have spaces between adjacent turns of the coil. This structure preferably exhibits a high degree of flexibility. Further, coiled layer 52 keeps bending portion 50 round when it is bent, preventing it from assuming an oval shape or another shape that could make it difficult to retract outer sheath 22 to deploy the prosthetic heart valve 20. As outer sheath 22 is retracted to deploy the prosthetic heart valve 20, internal friction will inhibit the retraction of coiled layer, causing braided layer 54 to lengthen. As the braided layer 54 lengthens, its diameter will collapse around coiled layer 52, allowing high tension forces without ovalizing even in a bend configuration. On the other hand, in the event a resheathing procedure becomes necessary, as the prosthetic heart valve is drawn into valve cover 56 (described below), the spaces between the turns of the coils in coiled wire will compress.

As depicted in FIG. 2, a steering catheter 24 may be concentrically nested within outer sheath 22 and is configured to be selectively curved to facilitate navigation through the patient's vasculature and portions of the heart. Steering catheter 24 may be formed from a stainless steel hypotube extending from a proximal section that is connected to steering catheter handle 14 to a distal section.

Although the particular nested configuration shown in FIG. 2 and described above represents one preferred embodiment for the various components of catheter assembly 16, alternative embodiments may include a different concentric arrangement of constituent parts. For example, some embodiments may combine steering catheter 24 and outer sheath 22 into one component and/or configure the outer sheath with steering functionality, some embodiments may include more than one catheter with steering functionality, etc.

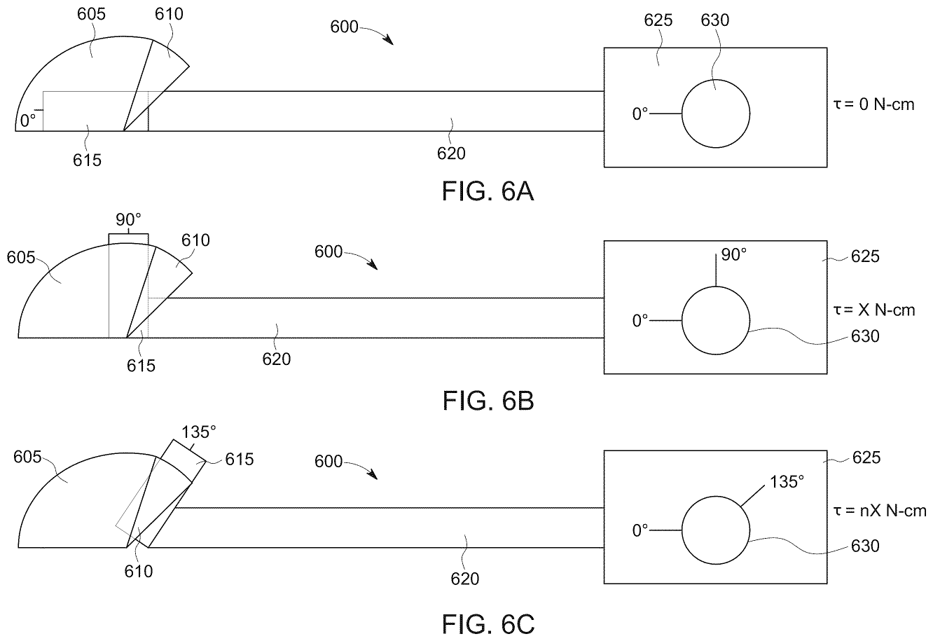

FIGS. 6A-C are schematic diagrams depicting a delivery system 600 in various states of deflection and schematically represent torque relative to deflection angle. As shown, the delivery system 600 can include a steerable catheter 620 having a flexible tip 615. The steerable catheter 620 and flexible tip 615 are steered by a steering mechanism 625, which may include a knob 630 to control deflection of the flexible tip 615 relative to a remainder of the steerable catheter 620. The steering mechanism 625 may include single-geared fixed-position radially-symmetric pulleys to apply tension to a cable that makes the flexible tip 615 of the steerable catheter 620 flex in the intended direction. In other words, rotating the knob 630 applies tension to a cable that connects the knob 630 to the flexible tip 615, and as that cable is tensioned via rotation of the knob 630, the flexible tip 615 deflects.

With this mode of actuation, the knob 630 experiences an increase in torque required to deflect the flexible tip as the tension on the pull-cable increases. The deflection angle begins at 0 degrees (e.g. straight relative to the remainder of the catheter 620) and increases as knob 630 is rotated (e.g., clockwise). The deflection angle can be in the range of 0 degrees to approximately 180 degrees, and the range of deflection angles may be thought of as having a first region 605 and a second region 610. The first region 605 generally includes angles not aligned with patient anatomy, while the second region 610 generally includes angles aligned with patient anatomy. In other words, angles within the first region 605 may not be anatomically relevant steering or deflection angles, while angles within the second region 610 may be anatomically relevant target deflection angles. In one example, the first region 605 is approximately 0 to 90 degrees while the second region 610 is approximately 90 to 180 degrees. In another example, the first region 605 is approximately 0 to 135 degrees while the second region 610 is approximately 135 to 180 degrees. In still another example, the first region 605 is approximately 0 to 145 degrees while the second region 610 is approximately 145 to 180 degrees.

By virtue of the single-geared fixed-position radially-symmetric pulleys, the knob 630 actuates the steerable catheter 620 at the same resolution irrespective of current deflection angle. In other words, the actuation resolution is the same in the first region 605, which may not be aligned with patient anatomy, as the second region 610, which may be aligned with patient anatomy. In this regard, actuation in the second region may be more challenging to achieve because of the increased torque due to the pull-cable tension. This torque can be represented as t=0 N*cm in FIG. 6A, where the actuation is at zero degrees. As the deflection angle is in the first region 605 and approaches the second region 610, the torque can be represented as t=X N*cm as shown in FIG. 6B, with X corresponding to a length of the pull cable. In the second region 610, the torque can be represented as T=nX N*cm, with n being a scalar value, as shown in FIG. 6C. As a result, the torque that must be applied to the control knob 630 to steer the flexible tip 615 through the anatomically relevant second region 610 is significantly greater than that which must be applied to the control knob 630 to steer the flexible tip through the less critical first region 605. This, in turn, can make it more difficult for the user to achieve the desired deflection of flexible tip 615 during a procedure, despite it being relatively easy to steer the flexible tip 615 through the less critical first region 605. Put even more simply, the non-linear torque of the steering of the delivery system 600 results in steering being user-friendly in the range where steering is not particularly important, and user-unfriendly in the critical range of steering that is relevant for the operative procedure. As one particular example of this, referring briefly back to FIG. 3, when a collapsible and expandable prosthetic mitral valve is transeptally delivered to a native mitral valve, the catheter tip (e.g. the valve cover 48) typically should be aligned so that it is coaxial with the longitudinal center of the native mitral valve. In order to achieve this coaxial position, the catheter tip must typically by angled a significant amount (e.g. at least 90 degrees) after passing through the atrial septum. Because achieving the coaxial alignment can be critical to procedural success, it would be desirable for the steering in that critical range to be easily controlled by the user.

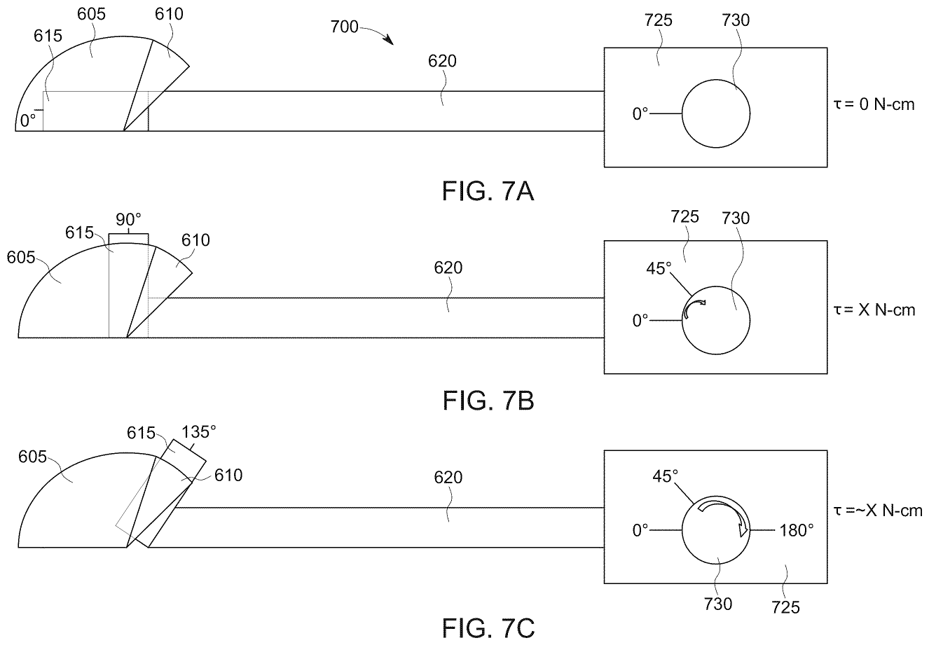

FIGS. 7A-C are schematic diagrams depicting a desired delivery system 700 in various states of deflection and schematically represents torque relative to deflection angle. In this example, the steering mechanism 725 can include a knob 730 and may be configured to provide a variable precision resolution in the first region 605 while providing a uniform torque in the second region 610.

As shown in FIG. 7B, the applied torque can be represented as t=X N*cm, which is the same or approximately the same as the applied torque in the example of FIG. 6B. Further, the knob 730 has been rotated by approximately 45 degrees in FIG. 7B, as compared to the approximately 90 degrees of rotation shown in FIG. 6B.

As shown in FIG. 7C, the applied torque can be represented as T=˜X N*cm, and can be the same or approximately the same as the applied torque in FIGS. 6B and 7B. This can be achieved by rotating the knob 730 from approximately 45 degrees to approximately 180 degrees.

Advantageously, the applied torque can be more uniform across the entire deflection angle of the flexible tip 615, especially in the second region 610 that aligns with patient anatomy, than the configuration depicted in FIGS. 6A-C. Further, the range of knob 730 rotation in FIG. 7C is greater than the range of knob 630 rotation in FIG. 6B, allowing for finer control of the flexible tip 615 in the second region 610.

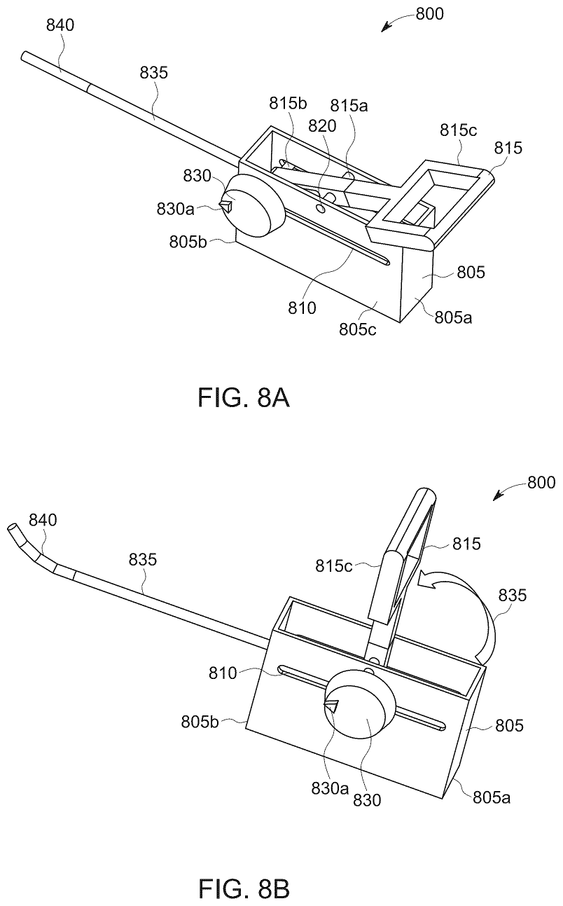

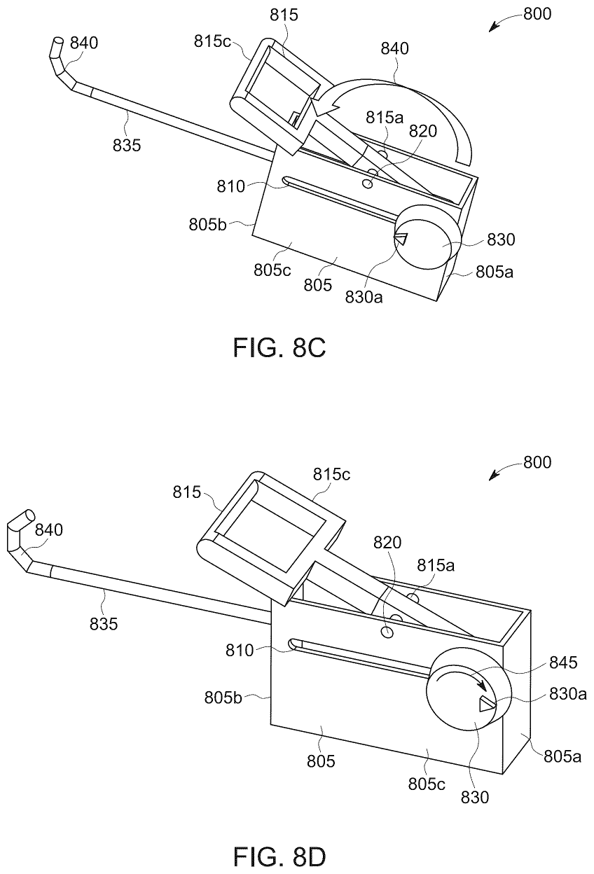

FIGS. 8A-D depict various stages of catheter deflection of a delivery mechanism 800 according to one or more aspects of the disclosure.

As shown, the delivery mechanism 800 can include a housing 805 having a first end 805a, a second end 805b, and lateral sides 805c, with the longitudinal axis of the housing 805 generally extending from second end 805b to first end 805a. The housing 805 can define a pair of longitudinal slots 810 formed in lateral sides 805c of the housing 805. Each of the pair of longitudinal slots 810 can be identical and can extend at least partially or completely between the first end 805a and the second end 805b. Preferably, the longitudinal slots 810 are positioned at about the same height relative to each other. In other examples, the pair of longitudinal slots 810 may not be identical, but may differ in one or more aspects. The pair of longitudinal slots 810 can be linear, substantially linear, or in other examples can be curved or curvilinear.

An actuator, such as lever 815 having a handle 815c, can be coupled to the housing 805 at a first coupling point 820 via first protrusions 815a. In other examples, the lever 815 can be any type of handle, slide, or a knob. The protrusions 815a can extend generally perpendicular to the longitudinal axis of the housing 805 and may be seated in holes defined by the housing 805 such that the protrusions 815a may rotate relative to the housing 805. Preferably, the protrusions 815a are fixed (e.g. rotationally fixed) relative to the lever 815, including via being formed integrally with the lever 815 (or a portion thereof). In this way, the lever 815 can be rotated or pivoted about the first coupling point 820, for example by manually pushing or pulling the lever 815 along the direction of the longitudinal axis of the housing 805. Although two protrusions 815a are shown as being received within two corresponding holes of the housing 805 at two coupling points 820, it should be understood that the lever 815 may be coupled to the housing 805 in other arrangements that still allow for the lever 815 to have the desired rotational movement upon pushing/pulling.

The lever 815 can also include second protrusions 815b which can extend generally perpendicular to the longitudinal axis of the housing 805. In the illustrated embodiment, the lever 815 includes two oppositely extending second protrusions 815b, each of which can be received within a corresponding one of the longitudinal slots 810 such that the second protrusions 815b can slide within the longitudinal slots 810 and along directions aligned with the longitudinal axis of the housing 805. With the slots 810 extending linearly as shown in FIGS. 8A-D, the protrusions 815b can be fixed to a further component that can slide into or out of the body of the lever 815 to accommodate the rotational arc of travel of the protrusions 815b.

During rotation or pivoting of the lever 815 about the first coupling point 820, the protrusions 815b can simultaneously slide within the longitudinal slots 810, such that the protrusions 815b are arranged near the second end 805b while the lever 815 is near the first end (and vice versa). A knob 830 may be rotatably attached to one of the protrusions 815b. During the rotation or pivoting of the lever 815, the knob 830 can also move longitudinally along (and/or relative to) the housing 805 generally along the path of the slots 810. The knob 830 can include a visual indicator 830a to indicate rotational position of the knob 830, as will be explained in greater detail below. In other examples, the knob 830 can be a rack and pinion style gear.

A steerable catheter 835 with a flexible tip 840 is coupled to a winding spool within the housing 805 such that rotation of the lever 815 and/or rotation of the knob 830 can actuate steering and control deflection angle of the flexible tip 840. In other words, a control wire (e.g. a steering wire/cable) may have a distal end coupled to the flexible tip 840, and may extend through the interior of the catheter (e.g. a steering lumen formed within a wall of the catheter 835), with a proximal end of the control wire coupled to the winding spool. Other components of the catheter 835 may not be directly coupled to the winding spool or any other components interior to the housing 805. In this regard, the lever 815 can act as a first control mechanism (e.g., a coarse control mechanism) that creates a bolus of tension (via linear actuation) on the steering cable of the steerable catheter 835 without collecting any further length of the steering cable on the winding spool. The knob 830 can act as a second control mechanism (e.g., a fine control mechanism) by collecting further length of the steering cable on the winding spool when the knob 830 is rotated (e.g. from an initial or unwound position shown in FIG. 8C to a further tensioned or wound position shown in FIG. 8D). One or both of the lever 815 or the knob 830 can be used to control deflection angle of the flexible tip 840 while allowing the user to effect two distinct deflection resolutions using the coarse control and the fine control.

FIG. 8A depicts the delivery mechanism 800 with the flexible tip 840 at 0 degrees of deflection relative to the remainder of the catheter 835. In this configuration, the lever 815 is unactuated and handle 815c is positioned near the first end 805a and the knob 830 is unactuated with the visual indicator 830a pointing toward the second end 805b.

FIG. 8B depicts the delivery mechanism 800 with the flexible tip 840 at some deflection angle between 0 and 90 degrees relative to the remainder of the catheter 835. In this configuration, the lever 815 is partially actuated (represented by rotational arrow 835) and thus handle 815c is mid-travel between the first end 805a and the second end 805b of the housing 805. This moves the winding spool (via translation of the winding spool, without rotation of the winding spool) from the second end 805b toward the first end 805a, thereby generating a bolus of tension on the steering cable extending through the steerable catheter 835 and deflecting the flexible tip 840. As also shown, while the knob 830 has slid from the second end 805b toward the first end 805a within the longitudinal slot 810 by virtue of rotation or pivoting of the lever 815, the knob 830 remains unactuated as the visual indicator 830a still points toward second end 805b.

FIG. 8C depicts the delivery mechanism 800 with the flexible tip 840 at approximately 90 degrees of deflection with respect to the remainder of the catheter 835. In this configuration, the lever 815 is completely actuated (represented by rotational arrow 840) and handled 815c is positioned near the second end 805b of the housing 805. This moves the winding spool toward the first end 805a, thereby generating a further bolus (as compared to the configuration of FIG. 8B) of tension on steering cable passing through the steerable catheter 835 and deflecting the flexible tip 840. As also shown, while the knob 830 has slid from the second end 805b toward the first end 805a within the longitudinal slot 810 by virtue of rotation or pivoting of the lever 815, the knob 830 remains unactuated as the visual indicator 830a still points toward second end 805b.

FIG. 8D depicts the delivery mechanism 800 with the flexible tip 840 at approximately 135 degrees of deflection relative to the remainder of the catheter 835. In this configuration, the lever 815 remains completely actuated and handle 815c is positioned near the second end 805b of the housing 805. No further bolus (as compared to the configuration of FIG. 8C) is generated by the lever 815. As also shown, the knob 830 remains near the first end 805a within the longitudinal slot 810, and the knob 830 has been actuated via rotation (represented by rotational arrow 845) and the visual indicator 830a is pointed towards the first end 805a. This rotation of the knob 830 (e.g., fine control mechanism) causes the steering cable passing through the steerable catheter 835 to wind up on the spool and thus further deflect the flexible tip 840.

While FIGS. 8A-8D depict the actuation of knob 830 only when the lever 815 is completely actuated, it is contemplated that the knob 830 can be actuated while the lever is in any state of actuation, including partially actuated or unactuated. Further, the knob 830 is depicted as actuating between the visual indicator 830a pointing toward the second end 805b and the first end 805a, it is contemplated that the rotational motion of the knob 830 can vary depending on the implementation.

Advantageously, the delivery system 800 provides for coarse control via the lever 815 and fine control via the knob 830. This allows for a more even application of torque across the various deflection angles (e.g., first region and second region) and allows for a finer resolution of deflection in the second region. It should be understood that the exact form of the housing 805, lever 815 and knob 830 may vary from that described above while still achieving the same goal of coarse deflection during the non-critical range of catheter tip deflection and fine deflection during the critical range of catheter tip deflection. For example, the housing 805, lever 815, and knob 830 may be provided as part of the handle assembly 14 of delivery system 10 shown in FIG. 1. In one example of use, a collapsible and expandable prosthetic mitral valve may be maintained within a collapsed condition within a valve cover operably connected to the flexible tip 840 (e.g. downstream of the tip so that deflection of the tip 840 causes corresponding positioning of the valve cover and the prosthesis contained therein). Just prior to, during, or just after the valve cover has passed through the atrial septum, the lever 815 may be actuated (up to and including full actuation) to sweep the catheter tip 840 through the first, non-critical range of deflection rather quickly and easily. Then, the knob 830 may be rotated to more precisely align the valve cover (via fine deflection of the catheter tip 840) to be coaxial with the native mitral valve annulus. As should be understood, the relatively unimportant first stage of deflection may be easily and quickly achieved, with the user able to control the relatively more important final stage of deflection highly accurately. As should further be understood, particularly in comparison to the embodiment of FIGS. 6A-C, the winding spool connected to the knob 830 may be at its minimum possible actuation when the critical deflection stage begins, helping to ensure that torque on the control knob 830 is relatively small when the critical deflection stage begins.

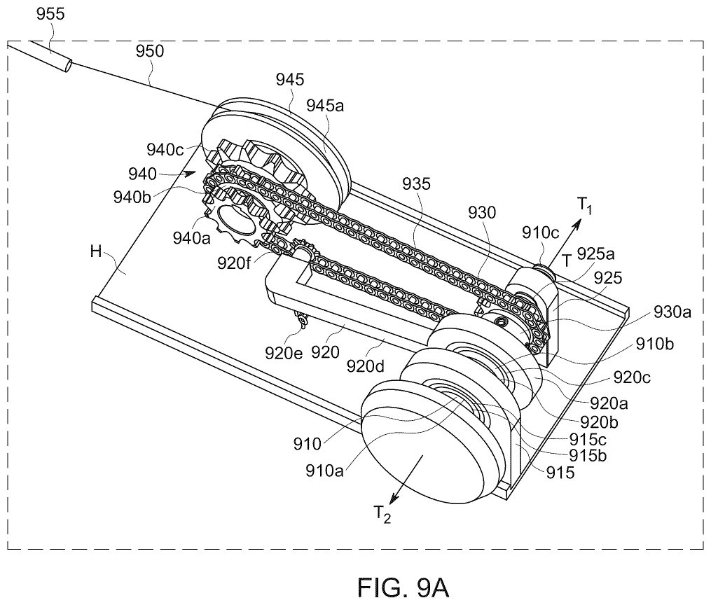

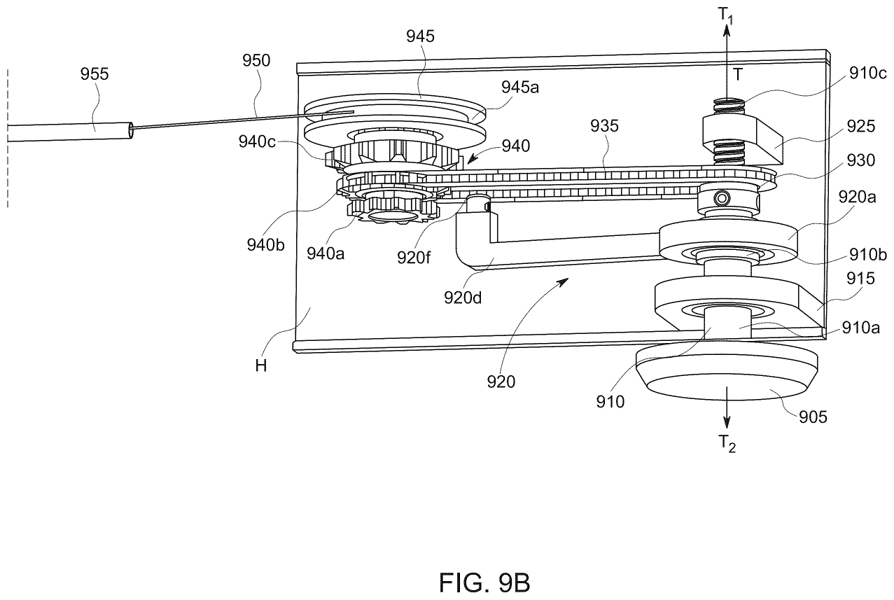

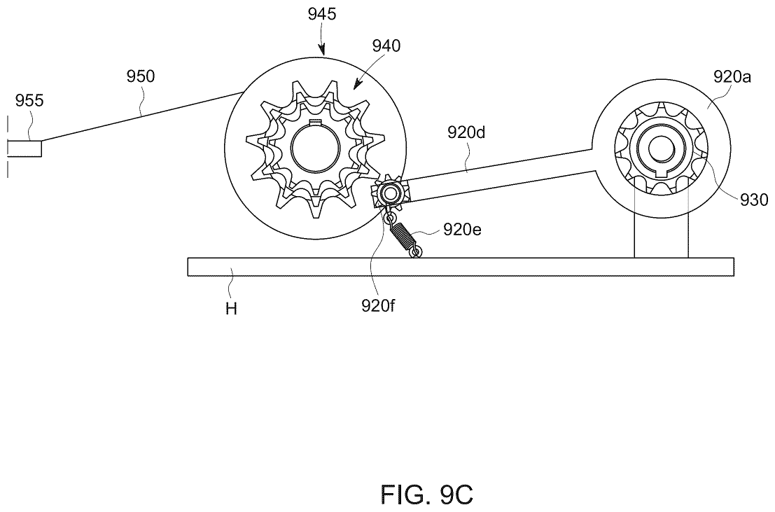

FIG. 9A is a perspective view of a portion of delivery system 900 and FIG. 9B is a top perspective view of the portion of delivery system 900 of FIG. 9A. FIG. 9C is a side cross-sectional view of the portion of delivery system 900 of FIG. 9A. As with system 800, components of system 900 may be incorporated into, for example, the handle 14 of delivery system 10 shown in FIG. 1.

As shown, the delivery system 900 can include a rotatable knob 905 that can be coupled to a drive gear 930 via axle 910. The axle 910 can be generally cylindrical and can define a translation axis T, which has a first translation direction T₁ and an opposite second translation direction T₂. As shown, the axle 910 can have a shaft 910a that defines a first diameter and one or more flanges 910b that extend radially outward from the shaft 910a and that define a second diameter greater than the first diameter. The axle 910 can also have a threaded portion 910c.

The knob 905 can be integrally formed with the axle 910 such that rotation of the knob 905 causes rotation of the axle 910. The threaded portion 910c can be positioned at an end of the axle 910 that is opposite the knob 905.

The shaft 910a can pass through a first support structure 915. The first support structure 915 can be coupled to a housing H (for example, an internal housing of handle 14 of delivery system 10 shown in FIG. 1) and can provide vertical support to axle 910. The first support structure 915 can define a hole 915b therethrough to receive the shaft 910a of the axle 910. In this regard, the diameter of the hole 915b can be greater a diameter of the shaft 910a. A bearing 915a can be positioned inside the hole 915b, for example between an inner diameter of the hole 915b of first support structure 915 and an outer surface of the shaft 910a, such that axle 910 can rotate freely relative to the first support structure 915 and the axle 910 can freely translate along the translation axis T relative to the first support structure 915. As shown in FIG. 9A, the axle 910 can translate along the translation axis T in either a first translation direction T₁ or a second translation direction T₂ that is opposed to T₁ based upon the rotation direction of the knob 905.

The axle 910 can include a plurality of flanges 910b that extend radially outward with respect to the shaft 910a such that the flanges 910b have a greater diameter than shaft 910a. As shown in FIG. 9D, there are two flanges 910b that are axially offset with one another, defining a portion of the shaft 910a therebetween. The portion of shaft 910a between the flanges 910b passes through a hole 920b of a coupling portion 920a of derailleur assembly 920. A bearing 920c can be positioned inside the hole 920b, for example between an inner diameter of the hole 920b of coupling portion 920a and an outer surface of the shaft 910a, such that axle 910 can rotate freely relative to the coupling portion 920a. The position of the flanges 910b on each axial side of coupling portion 920a allows for the axle 910 and the coupling portion 920a translate axially together along the translation axis T. While the axle 910 can rotate freely with respect to coupling portion 920a, a spring 920e can be attached at one side to the housing H and at another side to the derailleur assembly 920 (e.g., derailleur arm 920d) such that the derailleur assembly 920 remains relatively rotationally stationary.

The axle 910 can have a threaded portion 910c having external threads. The threaded portion 910c can pass through a hole 925a of a second support structure 925. The second support structure 925 can be coupled with the housing H and can provide vertical support to axle 910. In this example, the threaded portion 910c has an external threading that threadedly engages with internal threads of hole 925a of second support structure 925 such that the axle 910 can translate along the translation axis T relative to the second support structure 925.

The derailleur assembly 920 can include a derailleur arm 920d, which can be coupled to the housing H via a spring connection 920e, as described above, and an idler 920f positioned at an end of the derailleur arm 920d. The derailleur arm 920d can extend radially outward from the coupling portion 920a such that the teeth of idler 920f couple with chain 935. The idler 920f remains relatively rotationally stationary with respect to the chain 935, but can translate with the axle 910 as will be explained below.

A drive gear 930 can be coupled to the axle 910 such that the axle 910 and the drive gear 930 are rotationally fixed relative to one another and rotate simultaneously. In one example, the drive gear 930 is mounted to the axle 910 by a collar 930a. A chain 935 can be engaged with teeth of the drive gear 930 such that rotation of the drive gear 930 drives the chain 935, and the chain 935 drives the at least one gear 940a-c of cassette 940.

The cassette 940 can have a plurality of gears 940a-c. The plurality of gears 940a-c can be fixed relative to one another such that the gears 940a-c can rotate simultaneously. The plurality of gears 940a-c can be concentrically arranged, with each of the plurality of gears 940a-c being laterally offset from one another. In one example, the plurality of gears 940a-c can include a first gear 940a, a second gear 940b, and a third gear 940c. While three gears are depicted in the example of FIGS. 9A-C, it is contemplated that fewer or additional gears can be implemented depending on the particular configuration. As shown, the third gear 940c can have a greater diameter than second gear 940b and first gear 940a, and the second gear 940b can have a greater diameter than first gear 940a. In some examples, the third gear 940c can have a greater number of gear teeth than second gear 940b and first gear 940a, and the second gear 940b can have a greater number of gear teeth than first gear 940a, although the number of gear teeth do not necessarily need to correspond to the size of the gear. In other examples, each of the gears 940a-c can have a gear ratio selected to correspond to the drive gear 930 and to facilitate rotation of spool 945.

The spool 945 can be coupled to the cassette 940 such that they are rotationally fixed relative to one another and rotate simultaneously. The spool 945 can define a slot 945a to collect cable 950 from a steerable catheter 955 having a flexible tip (not shown). The cassette 940 and the gears 940a-c are rotationally fixed relative to the spool 945 such that rotation of the gears 940a-c causes rotation of the spool 945. A cable 950 can be wound around the spool 945. The cable 950 may be a steering wire or cable that passes through the steerable catheter 955 (e.g. through a steering lumen formed within a wall of the steerable catheter 955), with a distal end of the cable 950 coupled to a steering ring or similar component of a flexible tip (not shown). As shown, rotation of the knob 905 can cause rotation of spool 945, with the amount torque being exerted on cable 950 depending on which gear 940a-c the chain 935 is engaged with, as will be explained greater detail below.

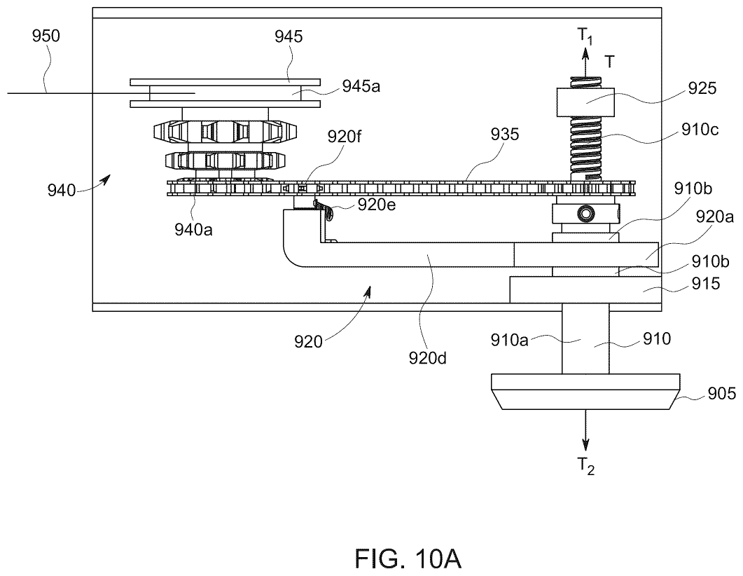

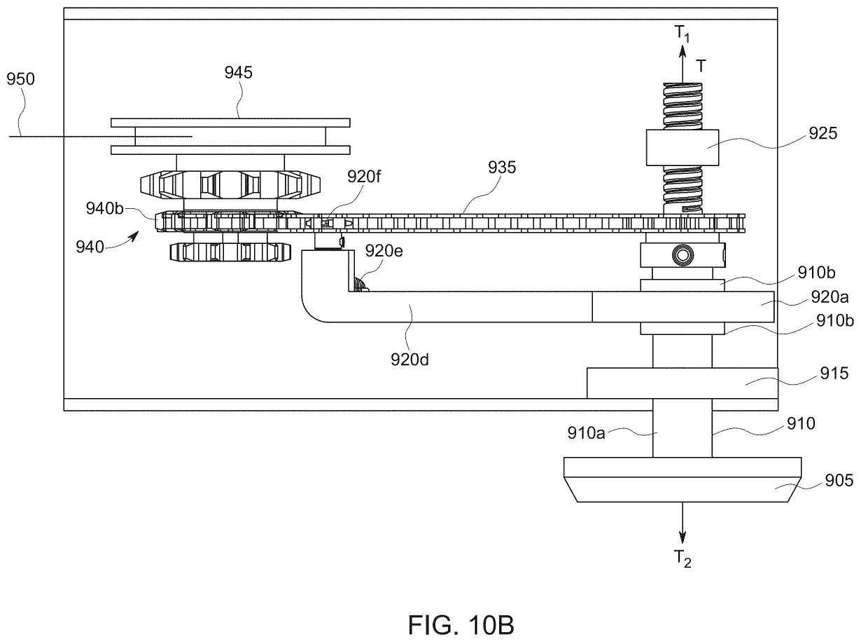

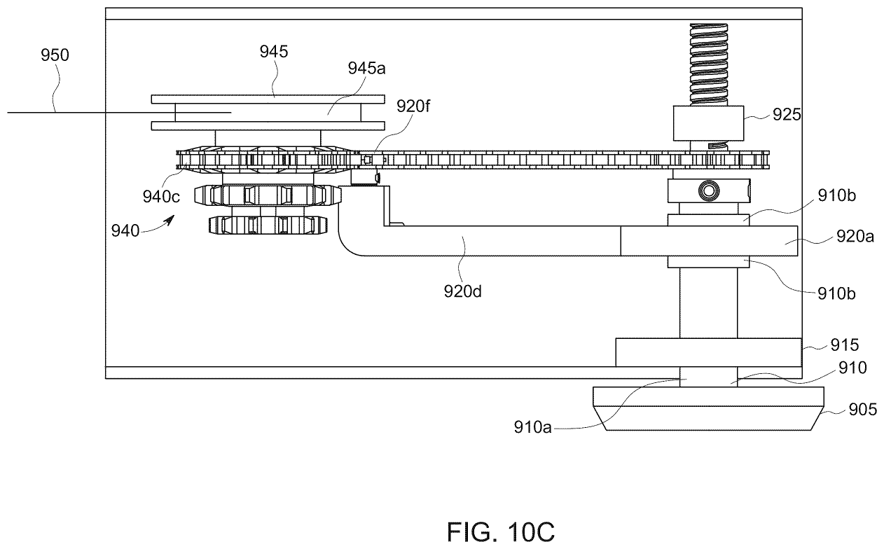

FIGS. 10A-C are top views of various stages of a steering operation of delivery system 900. FIG. 10A depicts a state in which the chain 935 is engaged with first gear 940a and with the flexible tip at some angle between 0 and 90 degrees. FIG. 10B depicts a state in which the chain 935 is engaged with second gear 940b and with the flexible tip at some angle between 90 and 120 degrees. FIG. 10C depicts a state in which the chain 935 is engaged with third gear 940b and with the flexible tip at some angle between 120 and 135 degrees. It should be understood that although the flexible tip is not actually shown in FIGS. 10A-C, the different configurations of FIGS. 10A-C nonetheless correspond to deflection ranges of the flexible tip expected in the illustrated configurations (e.g., FIGS. 7A-C).

Turning to FIG. 10A, the flanges 910b and the derailleur assembly 920 are axially closest to the first support structure 915. The axle 910 is prevented from translating further in the T₂ direction, since the flange 910b has a diameter greater than the diameter of the hole 915b defined by the first support structure 915.

In operation, the user can operate the knob 905 by rotating the knob 905 either clockwise or counterclockwise along arrow R. This causes the axle 910 to translate in the first translation direction T₁ along the translation axis T by virtue of threaded engagement between external threading on threaded portion 910c and internal threads of second support structure 925. During this translation, the coupling portion 920a and the axle 910 translate simultaneously by virtue of the flanges 910b that axially surround coupling portion 920a.

When the flexible tip is at some angle between 0 and 90 degrees relative to the remaining portion of the steerable catheter 965, the chain 935 is mechanically coupled with the first gear 940a. Upon rotation of the knob 905, the drive gear 930 correspondingly rotates by virtue of axle 910 and the axle 910 translates along the translation axis T in the first translation direction T₁. Rotation of the knob 905 also causes the steering cable 950 to collect on spool 945, creating tension on the flexible tip (for example via the connection between the steering cable 960 and the steering ring fixed to the flexible tip) and ultimately causing deflection of the flexible tip. In this configuration, a first torque is applied to cable 950 that is proportional to a radius of third gear 940a and applied force.

Upon continued rotation of the knob 905, the translation of the axle 910 in the first translation direction T₁ along the translation axis T continues until the idler 920f aligns with the second gear 940b, at which point the idler 920f can move the chain 935 from the first gear 940a to the second gear 940b.

Turning to FIG. 10B, the chain 935 has shifted from the first gear 940a to the second gear 940b and the flanges 910b and the derailleur assembly 920 have advanced axially away from the first support structure 915 (relative to the state depicted in FIG. 10A).

In this configuration, the flexible tip reaches an angle of about 90 degrees relative to the remainder of the steerable catheter 955. In this configuration, a second torque is applied to cable 950 by virtue of a radius of second gear 940b, with the second torque being greater than the first torque.

Upon continued rotation of the knob 905, the translation of the axle 910 in the first translation direction T₁ along the translation axis T continues until the idler 920f aligns with the third gear 940c, at which point the idler 920f can move the chain 935 from the second gear 940b to the third gear 940c.

Turning to FIG. 10C, the chain 935 has shifted from the second gear 940b to the third gear 940c and the flanges 910b and the derailleur assembly 920 have advanced axially away from the first support structure 915 (relative to the state depicted in FIG. 10B).

In this configuration, the flexible tip reaches an angle of about 120 degrees relative to the remainder of the steerable catheter 955. In this configuration, a third torque is applied to cable 950 by virtue of a radius of third gear 940c, with the third torque being greater than the first and second torque. In this regard, the delivery 900 is configured to deliver at least three distinct torques, thereby providing a variable torque over the range of deflection angles of the flexible tip.

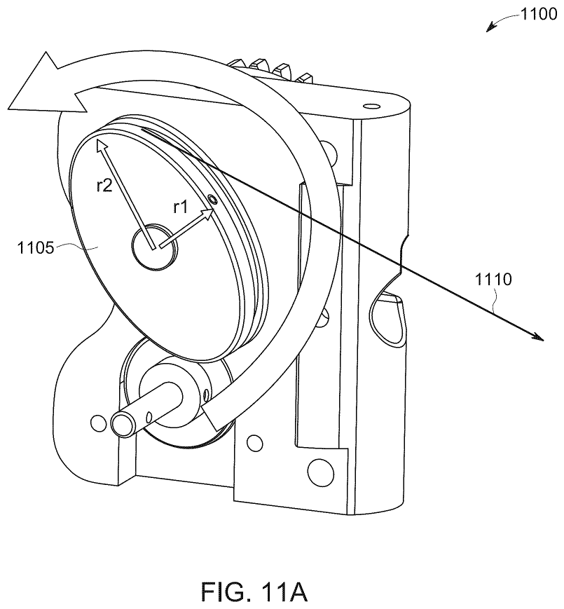

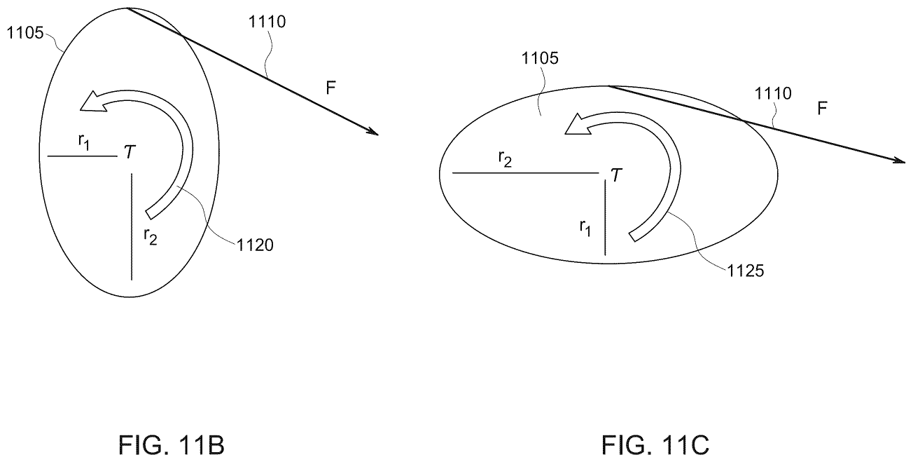

FIG. 11A is a perspective view of a portion of delivery system 1100. FIGS. 11B-C are schematic representations of exerted torque of the delivery system 1100. As with systems 800, 900, components of system 1100 may be incorporated into, for example, the handle 14 of delivery system 10 shown in FIG. 1.

The delivery system 1100 can include a spool 1105 that can collect a cable 1110. The delivery system 1100 can further include a knob for actuation, and the cable 1110 can be operatively connected to a steerable catheter. For example, the cable 1110 may be a steering cable that extends through the steerable catheter (e.g. through a steering lumen in a wall thereof) and couples to a flexible tip of the steerable catheter (e.g. via a steering ring).

The spool 1105 can any shape, such as any type of curved shape composed or not composed of circular arcs, that defines at least two distinct radiuses. For example, the spool 1105 can be elliptical such that it defines a semi-minor axis and a semi-major axis, thus defining at least two distinct radiuses. In other examples, the spool 1105 can be lens-shaped or oval-shaped. In still other embodiments, the spool 1105 can be cam-shaped so that there is a gradual increase in radius along nearly the entire circumference of the spool, and then a sharp drop back to the initial minimum radius. As shown in FIG. 11A, the spool 1105 is an oval having radiuses r1 and r2, with r2>r1. The spool 1105 can rotate (e.g., by operation of a knob) to collect cable 1110 operatively connected to a steerable catheter, thereby applying tension to the cable 1110 and deflecting a flexible tip of the steering catheter.

FIGS. 11B-C are schematic representations of exerted torque of the delivery system 1100, with FIG. 11B depicting torque exerted in a first region and FIG. 11C depicting torque exerted in a second region. As shown in FIG. 11B, a torque required to rotate (e.g., rotational arrow 1120) the spool 1105 to collect cable 1110 is proportional to r2 at the point of contact between cable 1110 and spool 1105. Upon continued rotation of the spool 1105 (e.g., rotational arrow 1125), the radius at the point of contact between cable 1110 and spool 1105 decreases, ultimately decreasing to r1. Correspondingly, the torque required to rotate decreases, since torque is proportional to radius. Thus, as radius decreases the required torque decrease and vice versa.

In operation, the configuration of FIG. 11B can represent a 0 degree deflection while the configuration of FIG. 11C can represent a maximum deflection of the flexible tip. As shown, the entire range of operation of the knob is 90 degrees, since rotation greater than 90 degrees results in the radius to increase from r1 back to r2. As the spool 1105 is rotated from the configuration in FIG. 11B to the configuration in 11C, the force required to generate torque continuously decreases by virtue of the continuously decreasing radius, resulting in a coarse control over non-critical angles of deflection and a fine control over the critical angles of deflection.

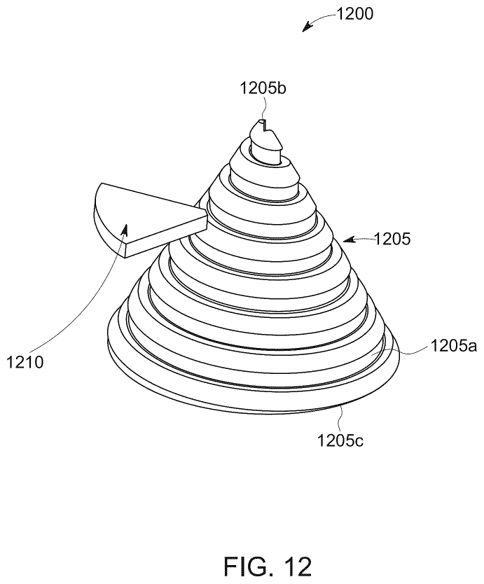

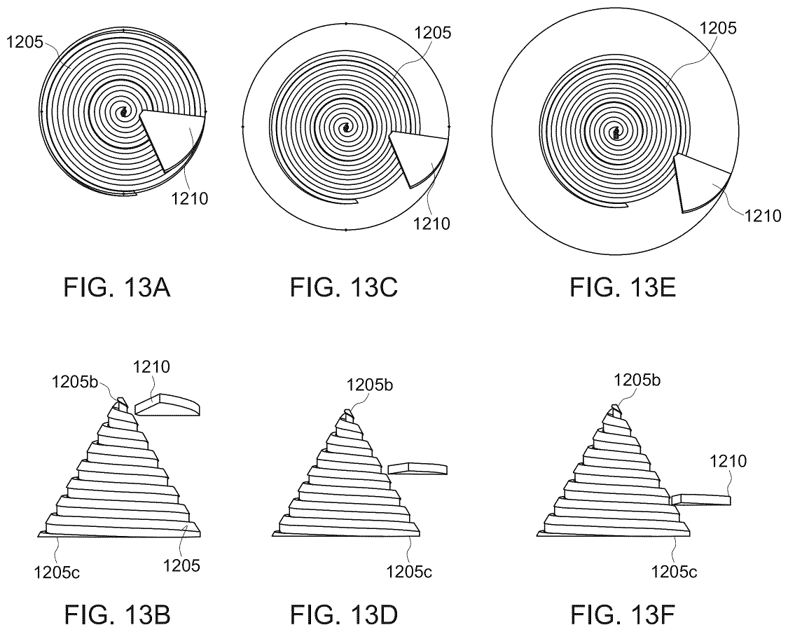

FIG. 12 is a perspective view of a portion of delivery system 1200. FIGS. 13A-F depict views of various stages of a steering operation of the delivery system 1200.

The delivery system 1200 can include a spool 1205 that can guide a cable and a follower 1210. The delivery system 1200 can further include a knob for actuation, a cable that can be collected on the spool 1205 and that can be operatively connected to a steerable catheter. The knob can be directly or indirectly coupled to the spool 1205 such that rotation of the knob causes rotation of the spool 1205. For example, the cable may be a steering cable that extends through the steerable catheter (e.g. through a steering lumen in a wall thereof) and couples to a flexible tip of the steerable catheter (e.g. via a steering ring).

As shown, the spool 1205 can be a conical cam and can define a path 1205a in the shape of a helix. The path 1205a can be a groove formed in the surface of the cam that can have a width and depth to accommodate a cable. The path 1205a can extend in a helical manner between a first end 1205b to a second end 1205c of the conical cam, with the radius of the conical cam increasing from the first end 1205b to the second end 1205c. In this regard, the conical cam can define a continuous range of radiuses from a smallest radius at first end 1205b to a largest radius at second end 1205c. The follower 1210 can be a wedge corresponding to an arc of a circle, and the cable can be fixed permanently or semi permanently to the follower 1210. The system 1200 can further include a hard stop, such as a plate, positioned at either or both of the first end 1205b and/or second end 1205c to prevent over-rotation or disengagement of the follower 1210 and cable relative to spool 1205.

As a knob is actuated, spool 1205 can be correspondingly rotated and follower 1210 can move toward or away from the spool 1205 and toward or away from either first end 1205b or second end 1205c depending on direction of rotation. As the follower 1210 moves from first end 1205b toward second end 1205c, the follower 1210 simultaneously moves away from spool 1205. The follower 1210 acts as a fixation/collection point for a cable/wire that is collected or advanced. As described above, a torque required to rotate the spool corresponds to a radius of the contact point. In this regard, the spool 1205 has a continuously increasing or decreasing radius depending upon collection point of the cable and thus has a continuously variable torque.

As shown in FIGS. 13A-B, the follower 1210 is positioned at nearest to first end 1205b at a region of smallest radius, thus resulting in the lowest torque required to collect cable. As the spool is rotated, as shown in FIGS. 13C-D, either or both of the spool 1205 or follower 1210 move such that the follower is moved towards the second end 1205c of the spool 1205 having greatest radius. This increases the torque required to collect cable. As the spool is further rotated, as shown in FIGS. 13E-F, either or both of the spool 1205 or follower 1210 move such that the follower 1210 is moved further towards the second end 1205c of the spool 1210 of greatest radius. This further increases the torque required to collect cable. Advantageously, the delivery system 1200 can exhibit a variable torque through the deflection angles of a flexible tip.

In operation, the configuration of FIG. 13C can represent a 0 degree deflection while the configuration of FIG. 13A can represent a maximum deflection of the flexible tip. As the spool 1205 is rotated from the configuration in FIG. 13C to the configuration in 13A, the force required to generate torque continuously decreases by virtue of the continuously decreasing radius, resulting in a coarse control over non-critical angles of deflection and a fine control over the critical angles of deflection.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

1. A medical device delivery system comprising:
a steering catheter having a flexible tip defining a deflection angle relative to the steering catheter; and
a delivery mechanism configured to control the deflection angle of the flexible tip, the delivery mechanism comprising a coarse control over a first range of deflection angles and a fine control over a second range of deflection angles.

2. The medical device delivery system of claim 1, wherein the coarse control comprises a lever configured to deliver a bolus of tension to the steering catheter.

3. The medical device delivery system of claim 2, wherein the fine control comprises a knob configured to rotate a spool and collect a cable of the steering catheter.

4. The medical device delivery system of claim 3, wherein the coarse control and the fine control operate independently.

5. The medical device delivery system of claim 3, wherein the delivery mechanism comprises:
a housing coupled to the lever by a first coupling point, the housing defining a pair of longitudinal slots configured to receive protrusions of the lever.

6. The medical device delivery system of claim 5, wherein manual actuation of the lever causes the knob to slide along a path defined by the longitudinal slots.

7. The medical device delivery system of claim 6, wherein the longitudinal slots are linear, curved, or curvilinear.

8. The medical device delivery system of claim 1, wherein the second range of deflection angles of the flexible tip aligns with a patient anatomy.

9. The medical device delivery system of claim 1, wherein the steering catheter is nested within a retractable outer sheath that is configured to deploy a prosthetic heart valve.

10. The medical device delivery system of claim 9, wherein the prosthetic heart valve is a prosthetic mitral valve.

11. A medical device delivery system comprising:
a steering catheter having a flexible tip defining a deflection angle relative to the steering catheter; and
a delivery mechanism configured to control the deflection angle of the flexible tip, the delivery mechanism comprising variable torque mechanism configured to deliver a first torque over a first range of deflection angles and second torque over a second range of deflection angles.

12. The medical device delivery system of claim 11, wherein the variable torque mechanism comprises a spool having a plurality of distinct radiuses.

13. The medical device delivery system of claim 12, wherein the spool is oval-shaped.

14. The medical device delivery system of claim 12, wherein the steering catheter is operatively connected to a cable, wherein cable is collected upon the spool and tension is applied to the cable, causing deflection of the flexible tip.

15. The medical device delivery system of claim 11, wherein the variable torque mechanism comprises:
a conical cam defining a helical path; and
a follower configured to act as a collection point for a cable relative to the helical path.

16. The medical device delivery system of claim 15, wherein the helical path defines a plurality of radiuses between a first end and a second end of the conical cam such that the variable torque mechanism is configured to exert a variable torque on the steering catheter.

17. The medical device delivery system of claim 16, wherein a cable is fixed permanently or semi-permanently to the follower such that the cable is collected or advanced relative to the conical cam, wherein the cable couples to the flexible tip.

18. The medical device delivery system of claim 17, wherein as the spool is rotated, either or both of the spool and/or the follower move such that the follower is moved toward the second end of the conical cam.

19. The medical device delivery system of claim 11, wherein the variable torque mechanism comprises:
an axle;
a drive gear coupled to the axle;
a derailleur assembly coupled to the axle; and
a cassette including a plurality of gears.

20. The medical device delivery system of claim 19, wherein the derailleur assembly includes an idler configured to move a chain from a first gear of the plurality of gears that applies the first torque to a second gear of the plurality of gears that applies the second torque.
